(19) United States
(12) Patent Application Publication
(10) Pub. No.: US 2021/0003565 A1
(43) Pub. Date: Jan. 7, 2021

(54) Dual channel immuno-quantitative test strip of Zearalenone-Deoxynivalenol

(71) Applicant: Jiangnan University, Wuxi (CN)

(72) Inventors: Xiulan SUN, Wuxi (CN); Jiadi SUN, Wuxi (CN); Jian JI, Wuxi (CN); Yinzhi ZHANG, Wuxi (CN); Miao LI, Wuxi (CN); Ying LIU, Wuxi (CN)

(21) Appl. No.: 16/991,111

(22) Filed: Aug. 12, 2020

(30) Foreign Application Priority Data
Aug. 14, 2019 (CN) 2019107489577

(51) Int. Cl. G01N 33/533; G01N 33/577; G01N 33/558

(52) U.S. Cl. CPC G01N 33/533; G01N 2469/20; G01N 33/558; G01N 33/577

(57) ABSTRACT

The disclosure relates to a zearalenone-deoxynivalenol dual-channel immunoquantitative test strip and belongs to the technical field of immunoassay rapid detection. The disclosure prepares fluorescent probes by labeling with fluorescent microspheres, including a fluorescent microsphere-zearalenone monoclonal antibody, a fluorescent microsphere-deoxynivalenol monoclonal antibody and a fluorescent microsphere-goat anti-mouse second antibody. A zearalenone artificial antigen, a fluorescent microsphere artificial antigen and a goat anti-mouse second antibody are respectively sprayed on a nitrocellulose membrane to serve as a detection line T1, a detection line T2 and a quality control line C to prepare the immunochromatographic test strip; a competitive immunoassay method is adopted, and zearalenone and deoxynivalenol in samples are quantitatively analyzed at the same time by reading fluorescence values of the detection lines on a fluorescence immunoanalyzer. This method for preparing fluorescent probes not only overcomes the shortcoming of difficult storage of colloidal gold in the test strip technology, but also is simple, efficient and high in sensitivity.

### TECHNICAL FIELD

The disclosure relates to a dual channel immuno-quantitative test strip of Zearalenone-Deoxynivalenol, and belongs to the technical field of immunoassay rapid detection.

### BACKGROUND

Zearalenone (ZEN), also known as F-2 toxin, is an estrogen-like mycotoxin produced by Fusarium graminearum, Fusarium tricinctum, Fusarium moniliforme and the like, is widely present in corn, wheat, sorghum and other grains and products thereof, has strong reproductive and developmental toxicity and teratogenic effects which can cause slow growth and immunosuppression of poultry and livestock, can enter the human body through the food chain to cause blood and immune toxicity, and has carcinogenic activity which can cause tumors and bring great harm to human health.

Vomitoxin, with the scientific name of deoxynivalenol (DON), is one of trichothecin olefins. Vomitoxin is a common mycotoxin, and the main objects to be polluted by vomitoxin are wheat, barley, oats, corn and other grain crops.

Grains and products thereof may be polluted by a plurality of toxin-producing mycotoxins during production, or infectious fungi can produce a plurality of toxins at the same time, so that grains and products thereof may contain more than one or more than one type of mycotoxins. 25% of the total global grain production is polluted by mycotoxins each year. All crops may be polluted by toxin-producing fungi during growth, post-harvest storage and processing, and contain biotoxins. It is urgent to develop a more sensitive and convenient method which can be used for rapid, high-throughput, multi-channel detection on site. At present, the main method for simultaneous detection of mycotoxins is HPLC-MS. However, due to the requirements of expensive equipment and complicated sample pretreatment, HPLC-MS is not suitable for rapid detection of mycotoxins in grains. Therefore, it is urgent to develop a method which can be used for simultaneous detection of multiple mycotoxins in a short time.

### SUMMARY

The disclosure provides a zearalenone-deoxynivalenol immunoquantitative test strip, the test strip includes a sample pad, a nitrocellulose membrane (NC membrane) and absorbent paper, the nitrocellulose membrane uses a zearalenone artificial antigen, a deoxynivalenol artificial antigen and a goat anti-mouse second antibody as a detection line T1, a detection line T2 and a quality control line C respectively, the dosage of the zearalenone artificial antigen is 0.2-1.6 μg/cm, and the dosage of the deoxynivalenol artificial antigen is 0.1-1.2 μg/cm.

In an embodiment of the disclosure, the distance between the three lines of the detection line T1, the detection line T2 and the quality control line C is 0.3-0.5 cm.

In an embodiment of the disclosure, the NC membrane is a cut strip with a width of about 0.3-1 cm, preferably 0.4 cm.

In an embodiment of the disclosure, according to the detection line T1, 0.2-0.8 mg/mL of the zearalenone artificial antigen is sprayed onto the NC membrane, and the spraying amount per centimeter of the NC membrane width is 1-2 μL. When the NC membrane width is 0.4 cm, the spraying amount is 0.4-0.8 μL.

In an embodiment of the disclosure, according to the detection line T2, 0.1-0.6 mg/mL of the deoxynivalenol artificial antigen is sprayed onto the NC membrane, and the spraying amount per centimeter of the NC membrane width is 1-2 μL. When the NC membrane width is 0.4 cm, the spraying amount is 0.4-0.8 μL.

In an embodiment of the disclosure, according to the quality control line C, 0.5-2 mg/mL of the goat anti-mouse second antibody is sprayed onto the NC membrane, and the spraying amount per centimeter of the NC membrane width is 1-2 μL/cm. When the NC membrane width is 0.4 cm, the spraying amount is 0.4-0.8 μL.

In an embodiment of the disclosure, the absorbent paper, the nitrocellulose membrane and the sample pad are successively adjacent to each other, and the length of the overlapped area of the adjacent parts is 2-4 mm.

In an embodiment of the disclosure, the overlapped part of the absorbent paper and the nitrocellulose membrane is located on the upper side of the nitrocellulose membrane.

In an embodiment of the disclosure, the test strip also includes a housing covering the test strip; the housing includes a base and a clamping shell, and the clamping shell is provided with an observation port and a sample inlet to expose a local area of the test strip; the sample inlet is formed in the upper part of the sample pad to expose part or all of the sample pad area; the observation port is formed in the upper side of the nitrocellulose membrane to expose the detection lines and the quality control line.

In an embodiment of the disclosure, a using method of the test strip includes:

(1) A zearalenone monoclonal antibody (Eu-ZEN-mAb) labeled with fluorescent microspheres (Eu) and a deoxynivalenol monoclonal antibody (Eu-DON-mAb) labeled with fluorescent microspheres (Eu) are uniformly mixed with a zearalenone-deoxynivalenol mixed standard and then added onto the sample pad in the above test strip for chromatography, and then an immunoquantitative analyzer is used for respectively measuring the corresponding fluorescence intensity values of T1 value, T2 value and C value of the mixed standard;

(2) A negative control is set, that is to say, a mixed standard does not contain zearalenone and deoxynivalenol, and the immunoquantitative analyzer is used for measuring the fluorescence intensity T₀ value;

(3) T1/T₀ and T2/T₀ are used respectively as parameters, and a linear model is established with a logarithmic value of concentration to obtain corresponding standard curves of zearalenone and deoxynivalenol;

(4) A to-be-tested sample is subjected to chromatography according to step (1) to obtain corresponding fluorescence intensity values, and the standard curves of the two toxins obtained in step (3) are respectively used to obtain content results of the toxins.

The disclosure also provides a method for dual-channel detection of zearalenone and deoxynivalenol by using the above test strip, and the method includes the following steps:

(1) A zearalenone monoclonal antibody (Eu-ZEN-mAb) labeled with fluorescent microspheres (Eu) and a deoxynivalenol monoclonal antibody (Eu-DON-mAb) labeled with fluorescent microspheres (Eu) are uniformly mixed with a zearalenone-deoxynivalenol mixed standard and then added onto the sample pad in the above test strip for chromatography, and then an immunoquantitative analyzer is used for respectively measuring the corresponding fluorescence intensity values of T1 value, T2 value and C value of the mixed standard;

(2) A negative control is set, that is to say, a mixed standard does not contain zearalenone and deoxynivalenol, and the immunoquantitative analyzer is used for measuring the fluorescence intensity T₀ value;

(3) T1/T₀ and T2/T₀ are used respectively as parameters, and a linear model is established with a logarithmic value of concentration to obtain corresponding standard curves of zearalenone and deoxynivalenol;

(4) A to-be-tested sample is subjected to chromatography according to step (1) to obtain corresponding fluorescence intensity values, and the standard curves of the two toxins obtained in step (3) are respectively used to obtain content results of the toxins.

In an embodiment of the disclosure, the chromatography time in step (1) is 10-15 min, preferably 10 min.

In an embodiment of the disclosure, the medium of the mixed standard is a PBS solution containing 10%-40% methanol and having the pH value of 7.4. The content of methanol is preferably 20%.

In an embodiment of the disclosure, 20-50 μL of to-be-tested sample, 40-50 μL of fluorescent microsphere-zearalenone antibody complex (Eu-ZEN-mAb) solution and 40-50 μL of fluorescent microsphere-deoxynivalenol antibody complex (Eu-DON-mAb) solution are mixed uniformly, 100-150 μL of the mixture is slowly dropped onto the sample pad of the test strip for chromatography at 35-37° C. for 10-15 min, then a T1 value, a T2 value and a C value are recorded by an HG-98 immunoquantitative analyzer, T/T₀ is used as a parameter, and quantitative detection of the sample is carried out. T refers to the fluorescence intensity at the line T1 or line T2 when different concentrations of samples are added; T₀ refers to the fluorescence intensity of a negative sample at a corresponding line T, and the negative sample refers to a sample without a target.

In an embodiment of the disclosure, a zearalenone complete antibody, a deoxynivalenol complete antibody and a goat anti-mouse monoclonal antibody are used in the form of a diluent, and the diluent is a PBS solution containing 1% BSA, 0.9% NaCl and 1% NaN3 and having the concentration of 0.01 M and the pH value of 7.4.

In an embodiment of the disclosure, an antibody complex solution (fluorescent probe) is a PBS solution containing 1% BSA and 0.05% Tween-20, wherein the concentration of the fluorescent probe is 0.25-5 μg/μg.

In an embodiment of the disclosure, zearalenone and deoxynivalenol standards are used in the form of a diluent, and the diluent is a PBS solution containing 20% methanol and having the concentration of 0.01 M and the pH value of 7.4.

In an embodiment of the disclosure, the dilution multiple of a fluorescent probe is 200.

In an embodiment of the disclosure, the feeding volume of a to-be-tested sample is preferably 40 μL.

In an embodiment of the disclosure, the feeding volume of a fluorescent microsphere-zearalenone antibody complex is 50 μL.

In an embodiment of the disclosure, the feeding volume of a fluorescent microsphere-deoxynivalenol antibody complex is 50 μL.

In an embodiment of the disclosure, the feeding volume of a mixture on the test strip is 140 μL.

In an embodiment of the disclosure, the time for chromatography at 37° C. is 10 min.

Compared with the prior art, the technical scheme of the disclosure has the following advantages:

(1) The method of the disclosure can realize dual-channel quantitative measurement of the content of zearalenone and deoxynivalenol, and the specificity and the sensitivity are high. When the concentration of zearalenone is 1 μg/L-10 μg/L, the logarithmic value of the concentration of zearalenone has a linear relationship with T/To, the linear equation is shown as Y=90.04-69.67 Log X, R²=0.9947, and the detection limit can reach 0.6903 μg/L; when the concentration of deoxynivalenol is 1 μg/L-25 μg/L, the logarithmic value of the concentration of deoxynivalenol has a linear relationship with T/To, the linear equation is shown as Y=72.90-20.64 Log X, R²=0.9974, and the detection limit can reach 0.2447 μg/L.

(2) The method of the disclosure uses the zearalenone monoclonal antibody and the deoxynivalenol antibody as identification targets, and the fluorescent microspheres are used as signal sources. The volume of the fluorescent microspheres is much larger than that of fluorescent dye molecules, the fluorescent microspheres cannot only load a large amount of the fluorescent dye molecules, but also facilitate the aggregation of the fluorescent dye molecules at the detection lines and the quality control line to play a role in signal amplification, the detection specificity is high, rapid quantitative detection can be achieved, the sensitivity is high, errors are small, and great convenience is provided for timely detection. The disclosure uses the fluorescent microspheres to replace colloidal gold and respectively uses the fluorescent microspheres to label the zearalenone antibody complex and the deoxynivalenol antibody complex. A competitive immunoassay method is used, and the complexes are used as a fluorescent probe for immunochromatography. By reading the fluorescence values of the detection lines on the fluorescence immunoanalyzer and quantitatively analyzing the zearalenone and deoxynivalenol in the sample at the same time, zearalenone and deoxynivalenol can be quickly and quantitatively detected.

(3) The test strip prepared by the disclosure is a rapid detection product which can truly meet market needs, has high precision and stability, simple operation and high repeatability, can meet the needs of industrial and commercial departments, quality inspection institutions, scientific research institutions and other testing institutions, can be used by various grain processing companies, third-party testing institutions, government supervision departments at all levels and the like, and is suitable for the fields of food industry, environmental protection, biochemistry and the like.

### BRIEF DESCRIPTION OF FIGURES

FIG. 1A refers to optimization of corresponding antigen concentrations of the deoxynivalenol detection line T2 in a zearalenone-deoxynivalenol dual-channel immunochromatography method; FIG. 1B refers to optimization of the corresponding antigen concentration of the zearalenone detection line T1 in a zearalenone-deoxynivalenol dual-channel immunochromatography method;

FIG. 2: Line T immunokinetic curves of a zearalenone-deoxynivalenol dual-channel immunochromatography method;

FIG. 3A refers to the influence of the content of methanol in a sample diluent on sample detection results (A); FIG. 3B refers to the influence of the content of methanol in a sample diluent on the inhibition rate of fluorescence intensity;

FIG. 4: A structural diagram of the zearalenone-deoxynivalenol dual-channel immunoassay test strip;

FIG. 5A is a standard curve of zearalenone for simultaneous detection of two toxins using labeled fluorescent microsphere immunochromatography; FIG. 5B is a standard curve of deoxynivalenol for simultaneous detection of two toxins using labeled fluorescent microsphere immunochromatography.

### DETAILED DESCRIPTION

Measurement of fluorescence intensity: An HG-98 immunoquantitative analyzer is used to measure corresponding fluorescence intensity values at the excitation wavelength of 365 nm and the emission wavelength of 613 nm.

### Example 1 Preparation of Fluorescent Probes of a Zearalenone Monoclonal Antibody Labeled with Eu-Fluorescent Microspheres and a Deoxynivalenol Monoclonal Antibody Labeled with Eu-Fluorescent Microspheres

A specific preparation method is as follows:

(1) 50 μL (1% solid content) of carboxyl fluorescent microspheres (purchased from Nanodot Technology in Xiamen, particle size 300 nm) placed at 4° C. are taken for ultrasonic dispersion, and 800-1000 μL of 2-(N-morpholine) ethanesulfonic acid (MES, C₆H₁₃NO₄S.H₂O) activation buffer with the concentration of 0.05 M is added for centrifugation at 14500 rpm for 10-15 min (the temperature during centrifugation is controlled to be about 15° C.);

(2) The supernatant is removed, 600-800 μL of MES buffer is added for ultrasonic resuspension, and repeat centrifugal cleaning is carried out 2-3 times;

(3) The supernatant is removed, 200 μL of MES buffer is added for ultrasonic resuspension, and 50 μL of 10 mg/mL 1-(3-dimethylaminopropyl)-3-ethylcarbodiimide hydrochloride (EDC, C₈H₁₇N₃.HCl) and 50 μL of 10 mg/mL N-hydroxysuccinimide (NHS, C₄H₅NO₃) are added into a shaker for oscillatory activation in the dark at 500 rpm at the room temperature (25° C.) for 30 min;

(4) Centrifugation is carried out, the supernatant is removed, and a phosphate buffer (PBS) with the concentration of 0.01 M and the pH of 7.4 is added for cleaning 2-4 times;

(5) The supernatant is removed, 400 μL of phosphate buffer is added for ultrasonic resuspension, a 10 μg of zearalenone monoclonal antibody (a preparation method reference: Sheng Jianwu, He Miao, Song Baodong, et al. Design and Preparation of MC-LR Complete Antigen [J]. Environmental Science, 2005, 26(3):33-37.), and oscillation is carried out in the dark for 2 h at room temperature;

(6) A 10% volume of 10 times blocking solution is added, and oscillation is carried out in the dark at room temperature for 30 min;

(7) Centrifugation is carried out after blocking, the supernatant is removed, and cleaning is carried out 2 times with a Tris-HCl (containing 0.1% Tween-20) buffer with the concentration of 0.05 M;

(8) The supernatant is removed, 200 μL of freeze-dried solution is added for redissolving to obtain a fluorescent probe of the zearalenone monoclonal antibody Eu-ZEN-mAb labeled with the Eu-fluorescent microspheres, and the fluorescent probe is stored at 4° C. for use.

According to the above steps, the zearalenone monoclonal antibody is replaced with the deoxynivalenol monoclonal antibody to prepare a fluorescent probe of the deoxynivalenol monoclonal antibody Eu-DON-mAb labeled with the Eu-fluorescent microspheres, and the fluorescent probe is stored at 4° C. for use.

### Example 2 Assembly of the Zearalenone-Deoxynivalenol Dual-Channel Immunoassay Test Strip Based on Labeling with the Fluorescent Microspheres

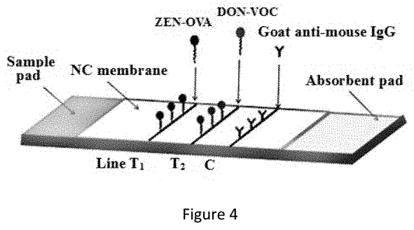

The structural diagram of the zearalenone-deoxynivalenol immunoassay test strip is shown as FIG. 4. There are the sample pad, the nitrocellulose (NC) membrane and the absorbent paper in order from left to right on a bottom plate. The key to the assembly of the test strip is to ensure consistent transferability between all parts, wherein the sample pad is stacked on the NC membrane with an overlapped part of about 5 mm. Similarly, the absorbent paper is stacked on the NC membrane with an overlapped part of about 5 mm. A cutting machine is used to cut a bonded plate into test strips with the width of about 4 mm, and the test strips are assembled with a plastic base and a clamping shell, sealed and stored at 4° C. for use.

An assembly method is as follows: the zearalenone complete antigen (0.2 mg/mL) and the deoxynivalenol complete antigen (0.3 mg/mL) are sprayed on the nitrocellulose membrane and used as the detection line T1 and the detection line T2 respectively, the goat anti-mouse second antibody (1 mg/mL) is respectively sprayed on the nitrocellulose membrane and used as the quality control line C, the spraying amount is 1 μL/cm, the distance between the three lines is about 5 mm, drying is carried out at 37° C. for 2-3 h, and the sprayed test strips should be cut and assembled in time, sealed and stored at 4° C. for use. The width of the lines T1, T2 and C depends on the diameter of a pipe of a membrane sprayer and is about 2 mm, and the length of the lines T and C, namely the width of a single test strip, is about 4 mm. The line T1 is about 5 mm away from the sample pad, the line T2 is about 10 mm away from the sample pad, and the line C is about 5 mm away from the absorbent paper.

### Example 3 Drawing of Standard Curves of the Zearalenone-Deoxynivalenol Immunoassay Test Strip Based on Labeling with the Fluorescent Microspheres

A drawing method of the standard curves is:

The zearalenone monoclonal antibody (Eu-ZEN-mAb) labeled with the Eu-fluorescent microspheres and the deoxynivalenol monoclonal antibody (Eu-DON-mAb) labeled with the Eu-fluorescent microspheres, which are prepared in Example 1, are used as fluorescent probes, 40 μL of mixed standard solution of zearalenone and deoxynivalenol, 50 μL of Eu-ZEN-mAb solution and 50 μL of Eu-DON-mAb solution are mixed uniformly, and 140 μL of the mixture is slowly dropped onto the sample pad of the test strip for chromatography at 37° C. for 10 min. Then the T value and the C value are recorded by an HG-98 immunoquantitative analyzer. The concentration gradients (ZEN/DON) of mixed standards of zearalenone and deoxynivalenol are 1/1 μg/L, 2/2 μg/L, 4/5 μg/L, 6/10 μg/L, 8/20 μg/L and 10/25 μg/L respectively. The logarithmic value of the concentration of each standard is used as the abscissa, and the T/T₀ (inhibition rate) value is used as the ordinate to draw a curve. T refers to the fluorescence intensity at the line T when different concentrations of samples are added, and T₀ refers to the fluorescence intensity of a negative sample at the line T; the mixed standard solution of zearalenone and deoxynivalenol contains different concentration gradients of zearalenone and deoxynivalenol, and a 0.01 M PBS solution containing 20% methanol (v:v) and with the pH of 7.4; the Eu-ZEN-mAb solution contains a 0.25 μg/μg fluorescent microsphere labeled ZEN monoclonal antibody, 0.1 g BSA and a 0.05 mL of 0.05 mol/L Tris-HCl solution of Tween-20 with the pH of 7.5; the Eu-DON-mAb solution contains a 0.25 μg/μg fluorescent microsphere labeled DON monoclonal antibody, 0.1 g BSA and a 0.05 mL of 0.05 mol/L Tris-HCl solution of Tween-20 with the pH of 7.5.

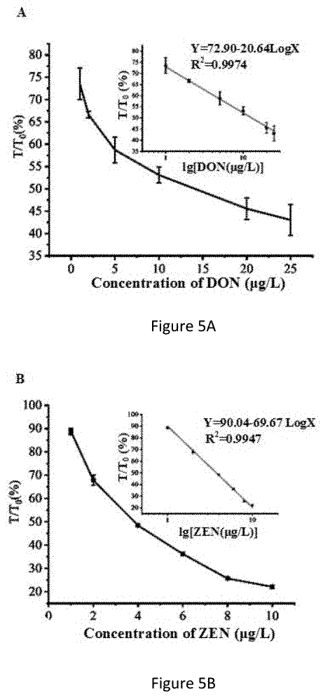

As the concentration of zearalenone and deoxynivalenol is increased, the lines T1 and T2 of the test strip become lighter and lighter, and T/T₀ becomes smaller and smaller. FIG. 5B is a T/T₀ curve changing with the concentration of zearalenone. When the concentration of zearalenone is 1 μg/L-10 μg/L, the logarithmic value of the concentration of zearalenone has a linear relationship with T/To, the linear equation is shown as Y=90.04-69.67 Log X, R²=0.9947, and the detection limit can reach 0.6903 μg/L. FIG. 5A is a T/T₀ curve changing with the concentration of deoxynivalenol. When the concentration of deoxynivalenol is 1 μg/L-25 μg/L, the logarithmic value of the concentration of deoxynivalenol has a linear relationship with T/To, the linear equation is shown as Y=72.90-20.64 Log X, R²=0.9974, and the detection limit can reach 0.2447 μg/L.

### Example 4 Optimization of Process Conditions for Simultaneous Rapid Detection of Zearalenone and Deoxynivalenol Based on Labeling with the Fluorescent Microspheres

(1) The Influence of the Dosage of the Zearalenone Artificial Antigen and the Deoxynivalenol Artificial Antigen

In order to improve the detection sensitivity of the test strip, the influence of different complete antigen concentrations during an experiment on the competitive inhibition rate of a negative control group and a sample with the zearalenone content of 4 μg/L is studied.

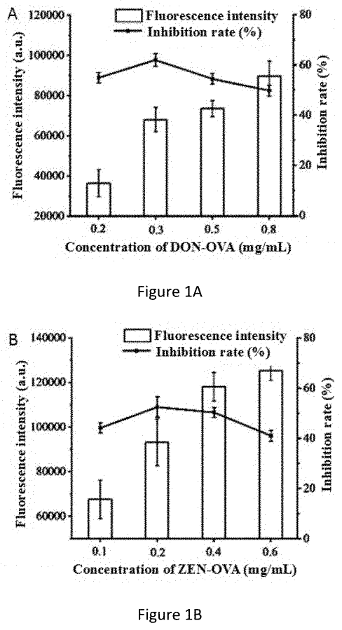

The specific experimental exploration process is as follows: When the zearalenone-deoxynivalenol dual-channel immunochromatographic test strip is prepared, in the process of spraying a DON-OVA artificial antigen at the detection line T1, the concentration of DON-OVA is selected as 0.2 mg/mL, 0.3 mg/mL, 0.5 mg/mL and 0.8 mg/mL, the concentration of ZEN-OVA sprayed at the detection line T2 is selected as 0.1 mg/mL, 0.2 mg/mL, 0.4 mg/mL and 0.6 mg/mL, the negative control group (PBS and a 20% methanol-PBS solution) and a positive test group (DON concentration is 5 μg/L, ZEN concentration is 4 μg/L) are selected for analysis, and then the influence of different concentrations of DON-OVA and ZEN-OVA on an immunochromatography method is evaluated. Results are shown in Table 1 and FIG. 1A, FIG. 1B:

TABLE 1
Optimization of the dosage of two antigens

| | Antigen | Inhibition rate | Fluorescence |
|---|---|---|---|
| | concentration | (%) | intensity (a.u.) |
| Deoxynivalenol | 0.2 | 56 | 38759 |
| artificial | 0.3 | 62 | 68973 |
| antigen | 0.5 | 55 | 74586 |
| | 0.8 | 50 | 90527 |
| Zearalenone | 0.1 | 45 | 67941 |
| artificial | 0.2 | 55 | 93573 |
| antigen | 0.4 | 52 | 119840 |
| | 0.6 | 42 | 125735 |

From Table 1 and FIG. 1A, it can be seen that as the concentration of the DON-OVA complete antigen at the line T1 is increased, the fluorescence intensity of the negative control group (PBS solution) at the detection line is first increased and then becomes stable; when the concentration of the DON-OVA complete antigen is 0.3 mg/mL, the competitive inhibition rate of the positive test group (DON=5 μg/L) and the negative control group reaches the maximum, and there is also a relatively high fluorescence signal value at the detection line at this time. Therefore, 0.3 mg/mL is taken as the optimal concentration of the DON-OVA complete antigen at the line T. From Table 1 and FIG. 1B, it can be seen that as the concentration of the ZEN-OVA complete antigen at the line T2 is increased, the fluorescence intensity of the negative control group (20% methanol-PBS solution) at the line T2 is first increased and then becomes stable; when the concentration of the ZEN-OVA complete antigen is 0.2 mg/mL, the competitive inhibition rate of the positive test group (ZEN=4 μg/L) and the negative control group reaches the maximum, and there is also a relatively high fluorescence signal value at the line T at this time. Therefore, 0.2 mg/mL is taken as the optimal concentration of the DON-OVA complete antigen at the line T.

(2) The Influence of Chromatography Time on Test Results:

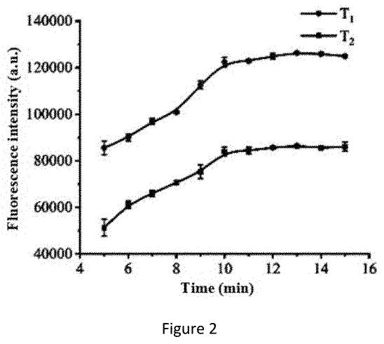

FIG. 2 is line T immunokinetic curves of the zearalenone-deoxynivalenol two-channel immunochromatography method. The experimental process is as follows: According to the process in Example 3, 40 μL of a to-be-tested sample, 50 μL of fluorescent microsphere-DON antibody complex and 50 μL of fluorescent microsphere-ZEN antibody complex are placed in a 96-well microwell plate, after a reaction for 5 min, 100 μL of the mixture is taken and added every 1 min onto the sample pad for chromatography for 5 min, and then changes in fluorescence intensity at the lines T1 and T2 are recorded by an HG-98 immunoquantitative analyzer.

The immunoreaction kinetic curves are drawn with the fluorescence intensity as the ordinate and the reaction time as the abscissa, changes of the fluorescence intensities of the two lines T with time are observed, and the time when the fluorescence values of the lines T1 and T2 become stable is used as the best detection time. It can be seen from FIG. 2 that the fluorescence intensities of the two lines T both show an increasing trend with time within 5-15 min. At the first 5 min of the reaction, the fluorescence intensity is weak, errors between parallel groups are large, and it is indicated that the reaction is not stable at this time; after the reaction is carried out for 10 min, the fluorescence intensity values of the two lines T are no longer changed significantly and become stable, and errors between the parallel groups are gradually reduced. Therefore, 10 min is selected as the pre-reaction time before a sample is added onto the test strip for immunochromatography.

(3) Optimization of the Concentration of Methanol in a Sample Diluent

In an immunochromatographic reaction, an antibody has certain tolerance to methanol, and a high concentration of methanol has a certain destructive effect on the antibody and even inactivates the antibody. An appropriate concentration of methanol has little effect on antibody activity, and the best detection effect can be obtained at the same time.

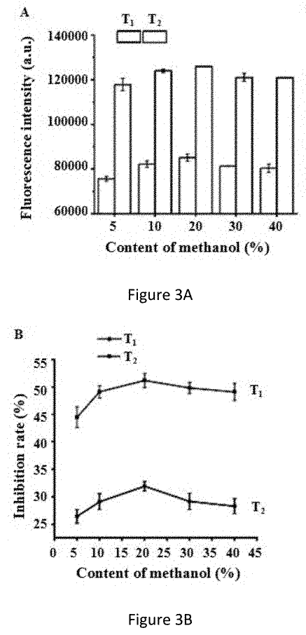

FIG. 3A and FIG. 3B shows the influence of methanol in a sample diluent on sample test results. In this experiment, the methanol volume concentration of a negative sample solution is respectively set to be 5%, 10%, 20%, 30% and 40%, all samples are spiked to 100/60 μg/L (DON/ZEN) with corresponding different contents of methanol solutions, the remaining process is similar to Example 3, and the immunochromatographic test strip is used for detection. The fluorescence intensities at T1 and T2 are recorded, and the influence of the concentration of methanol in a sample diluent on the inhibition rate of a positive sample (sample with spike) and a negative sample (sample without a target) is analyzed. It can be seen from FIG. 3 A and FIG. 3B that the concentration of methanol in the sample diluent has little influence on the fluorescence intensities of the two detection lines, but has significant influence on the inhibition rate. When the content of methanol is 20%, the inhibition rate of DON and ZEN in a sample and the negative control group is the largest. When the content of methanol is higher than 20%, the inhibition rate is significantly reduced, and the influence of the content of methanol in a sample diluent on the inhibition rate of ZEN is greater.

### Example 5 Evaluation of Accuracy of the DON-ZEN Dual-Channel Immunochromatographic Test Strip

In order to verify the accuracy and sensitivity of the dual-channel immunochromatographic test strip, spike and recovery experiments are carried out on negative wheat flour samples and corn flour samples. A high spike concentration, a medium spike concentration and a low spike concentration are set for the addition concentration of each sample, each group of concentration gradients have three groups of parallel experiments. According to a calculation formula of a spike and recovery rate, the spike and recovery rate of each sample is calculated to evaluate the accuracy of the method. The calculation formula of the spike and recovery rate is as follows:

$$spike and recovery rate ( \% ) = \frac{detected standard concentration}{added standard concentration} \times 100$$

The negative wheat flour samples and the corn flour samples are subjected to DON-ZEN spike and recovery experiments according to three concentration gradients of 50/20 μg/L, 100/60 μg/L and 200/80 μg/L respectively. In this study, a PBS solution containing 20% methanol and 0.05% Tween-20 is used as a sample diluent to ensure the performance of the method. A 5.0 g of a sample is accurately weighed, 25 mL of ultrapure water is added for vortex oscillation of the sample, after ultrasonic extraction for 30 min, centrifugation is carried out at 5000 rpm for 15 min, and the supernatant is filtered with a 0.22 μm filter membrane and then diluted 10 times with PBST before being used in immunochromatography detection.

Because a 80% methanol solution (methanol: water=80:20) is used to extract DON and ZEN in an actual sample during sample pretreatment, a extract is diluted 5 times with a sample diluent (PBST diluted extract) before detection to reduce the matrix effect of the sample and the influence of methanol in the extract on the detection accuracy. (Sample extract 1 μg/L=actual sample 10 μg/L)

TABLE 2
Evaluation of accuracy

| | DON-ZEN | | | |
|---|---|---|---|---|
| | mixed | | CV (%) | |
| | standard | Recovery rate | In | Out of |
| Samples | (μg/L) | (%, DON/ZEN) | batch | batch |
| Wheat | 50/20 | 92.71 ± 6.02/84.87 ± 3.98 | 6.23 | 11.39 |
| flour | 100/60 | 80.14 ± 8.90/88.64 ± 7.22 | 7.21 | 10.80 |
| | 200/80 | 87.42 ± 11.03/91.24 ± 6.53 | 10.74 | 7.98 |
| Corn | 50/20 | 105.73 ± 6.55/91.43 ± 8.20 | 7.35 | 8.93 |
| flour | 100/60 | 87.39 ± 7.30/92.42 ± 7.91 | 6.77 | 12.84 |
| | 200/80 | 89.52 ± 3.55/101.70 ± 6.32 | 5.83 | 6.52 |

It can be seen from Table 2 that the prepared DON-ZEN dual-channel detection immunochromatographic test strip is used to detect the spiked wheat and corn samples, the finally obtained spike and recovery rate of wheat is in the range of 80.14%-92.71%, the spike and recovery rate of corn is in the range of 87.39%-105.73%, and it is indicated that the prepared DON-ZEN dual-channel fluorescent immunoquantitative test strip can be used for rapid on-site screening and detection.

1. A zearalenone-deoxynivalenol dual-channel immunoquantitative test strip, comprising a sample pad, a nitrocellulose membrane and absorbent paper, wherein the nitrocellulose membrane comprises a zearalenone artificial antigen, a vomitoxin artificial antigen and a goat anti-mouse second antibody to be used as a detection line T1, a detection line T2 and a quality control line C, respectively, wherein the zearalenone artificial antigen is 0.2-1.6 μg/cm, and the deoxynivalenol artificial antigen is 0.1-1.2 μg/cm.

2. The test strip according to claim 1, wherein a distance between the three lines of the detection line T1, the detection line T2 and the quality control line C is 0.3-0.5 cm.

3. The test strip according to claim 1, wherein the absorbent paper, the nitrocellulose membrane and the sample pad are successively adjacent to each other; and a length of an overlapped area of adjacent parts is 2-4 mm.

4. The test strip according to claim 2, wherein the absorbent paper, the nitrocellulose membrane and the sample pad are successively adjacent to each other; and a length of an overlapped area of adjacent parts is 2-4 mm.

5. A method of using the test strip according to claim 1 to perform dual-channel detection of zearalenone and deoxynivalenol, comprising the following steps:
(1) uniformly mixing a zearalenone monoclonal antibody Eu-ZEN-mAb labeled with fluorescent microspheres and a deoxynivalenol monoclonal antibody Eu-DON-mAb labeled with fluorescent microspheres with a zearalenone-deoxynivalenol mixed standard and then adding onto the sample pad in the test strip according to claim 1 for chromatography, and then using an immunoquantitative analyzer to separately measure corresponding fluorescence intensity values of T1 value, T2 value and C value of the mixed standard;
(2) setting a negative control, namely, the mixed standard not containing zearalenone and deoxynivalenol, and using the immunoquantitative analyzer to measure a fluorescence intensity T0 value;
(3) separately using T1/T₀ and T2/T₀ as parameters, and establishing a linear model with a logarithmic value of concentration to obtain corresponding standard curves of zearalenone and deoxynivalenol; and
(4) performing chromatography on a to-be-tested sample according to step (1) to obtain corresponding fluorescence intensity values, and separately using standard curves of two toxins obtained in step (3) to obtain content results of the toxins.

6. The method according to claim 5, wherein the chromatography time in the step (1) is 10-15 min.

7. The method according to claim 5, wherein a medium of the mixed standard is a PBS solution containing 10%-40% methanol.

8. The method according to claim 6, wherein a medium of the mixed standard is a PBS solution containing 10%-40% methanol.

9. The method according to claim 5, wherein an antibody complex solution is a PBS solution containing 1% BSA and 0.05% Tween-20, and wherein a concentration of a fluorescent probe is 0.25-5 μg/μg.

10. The method according to claim 6, wherein an antibody complex solution is a PBS solution containing 1% BSA and 0.05% Tween-20, and wherein a concentration of a fluorescent probe is 0.25-5 μg/μg.

11. The method according to claim 7, wherein an antibody complex solution is a PBS solution containing 1% BSA and 0.05% Tween-20, and wherein a concentration of a fluorescent probe is 0.25-5 μg/μg.

12. The method according to claim 5, wherein the chromatography time in the step (1) is 10 min.

13. The method according to claim 5, wherein a medium of the mixed standard is a PBS solution containing 20% methanol.
